(19) United States
(12) Patent Application Publication
(10) Pub. No.: US 2023/0321393 A1
(43) Pub. Date: Oct. 12, 2023

(54) SYSTEM AND METHOD FOR DELIVERING TREATMENT

(71) Applicant: Lee-Anne THOMS, West Bridgewater, MA (US)

(72) Inventor: Lee-Anne THOMS, West Bridgewater, MA (US)

(21) Appl. No.: 18/295,758

(22) Filed: Apr. 4, 2023

Related U.S. Application Data
(60) Provisional application No. 63/328,613, filed on Apr. 7, 2022.

(51) Int. Cl. A61M 21/02; G16H 20/30

(52) U.S. Cl. CPC A61M 21/02; G16H 20/30; A61M 2230/50; A61M 2230/04; A61M 2230/30

(57) ABSTRACT

System and method for delivering a therapy treatment to help people release emotions and distress attached to disturbing memories. The system and method can be used in conjunction with web-based or mobile-based applications to provide horizontal eye movement or bilateral auditory or tactile stimulation to process out the emotional disturbances, specifically, using the application together with headphones, light bars or tactile objects. In some instances, sensors and monitors including blood pressure, heart rate, breathing rate and body temperature may be incorporated in conjunction with tactile, visual or auditory devices.

### CROSS REFERENCE TO RELATED APPLICATIONS

This application claims benefit from U.S. Provisional Patent Application Ser. No. 63/328,613, filed Apr. 7, 2022, which is hereby incorporated by reference in its entirety herein.

### FIELD OF THE INVENTION

The present invention relates generally to mental health treatments and, more specifically, to mental health treatments directed to overcoming trauma and distressing life experiences.

### BACKGROUND

Various mental health treatments exist today. For example, Eye Movement Desensitization and Reprocessing (EMDR) is a psychotherapy treatment that may help people release emotions and sensations attached to distress and disturbing memories. EMDR may be helpful in the treatment of post-traumatic stress disorder (PTSD). EMDR may be adapted for special needs population such as those with autism, a complex neurodevelopmental disorder, among others with traumatic or disturbing memories or experiences. For those with autism, co-morbid symptoms in autism spectrum disorder (ASD) can include language and learning disorders, anxiety, attention deficit hyperactivity disorder (ADHD) and obsessive-compulsive disorder (OCD), sensory processing disorders, disruptive, impulsive and conduct disorders, complex medical issues, sleeping problems, and immune and gastrointestinal dysfunction, among other symptoms and disorders. Due to the wide variety of symptoms and conditions, new and improved mental health treatments are needed.

### SUMMARY

The following presents a simplified summary of the innovation to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

In one embodiment, the system and method are to facilitate therapy treatment via an improved process, which can be based on an adaptive information processing model using eye movements of bilateral stimulation (BLS) to process inherent information in the neural memory networks. BLS of the brain can be accomplished by either through horizontal eye movement of bilateral auditory or tactile stimulation to process the emotional disturbances. As examples and by no ways limiting, the disclosed system and method of treatment may be used on athletes and lay person alike, or by speech therapists for treatment of others. Additionally, BLS may include other stimuli including visual, auditory and tactile, and can occur in rhythmic left-right pattern. In the alternative, the BLS need not be rhythmic or have any left-right patterns. Other examples of BLS may include visual stimuli by watching a hand or moving light alternating from left to right, and back again, or auditory stimuli by listening to tones that alternate between left and right sides of the head. In some instances, the therapy treatment can target disturbing memories. In other instances, the therapy treatment may help people rest and relax, with or without any physical and mental ailments, for calming the recipient of the treatment and helping to regulate their mental processes.

Disclosed herein are different ways of providing and delivering an improved EMDR-like therapy to patients based on new research and approach. The disclosed system and method can be implemented with a web-based application accessible via laptops or mobile devices, or with a mobile-based application accessible on mobile devices such as cell phones and portable tablets. The disclosed system and method can provide auditory stimuli to a recipient, where the auditory stimuli can alternate between left and right sides of the head.

In some embodiments, the disclosed system may be implemented via a software application that is configured to run on a mobile phone or a tablet device. The application can be configured to run natively on the device. Using the native software development kits (SDKs), the application can be created to provide a graphical user interface (GUI) to display the application screens. Alternatively, the application could be a mobile web application or a cross-platform application.

In some embodiments, a treatment method is as follows: To begin, after downloading installing the application, a user opens the application. In the alternative, a user may access the application via a web browser without installing the application. Login information may or may not be required depending on if the user is accessing the application with his/her own personal device (e.g., automatic login) or if the user is accessing the application via the Internet. Ways of logging in and setting up an account can use known steps in the art and will not be elaborated further herein. In some embodiments, a licensed medical professional such as a doctor or a therapist can set up a user account on the application having multiple patient profiles. In the alternative, a patient can set up his/her own user profile for personal use, or a guardian or parent can set up a user account for the treatment of his/her ward or child.

In one embodiment, a therapy treatment method includes initially providing an application, where the application can be web-based or mobile-based. The application can include at least one controller for manipulating an electronic device. For example, the controller can be used to control sound, frequency, tones, volume, visual or auditory stimulations. The electronic device capable of being controlled by the controller of the application can include headphone, light bar or tactile objects. In some instances, the electronic device can include blood pressure, heart rate, breathing rate and body temperature sensors and monitors. The external device can be in communication with the application, whether via a wired connection or a wireless connection (e.g., Wi-Fi connection).

In operation, a user can trigger the application to deliver a stimulus that is associated with the electronic device. For example, if the electronic device is a headphone the trigger can be frequency, tone, interval, or volume to the headset. In the alternative, if the electronic device is a light bar or a tactile object, the application can trigger lights at different intervals/intensity to be delivered by the light bar, or initiate vibrations at different intervals/intensity to the tactile object.

Next, the user analyzes the response from the patient based on the stimulus from the electronic device. The response can be done by examining the patient or by hooking up the patient to external monitors and watching for the patient's brain response depending on the type of stimulus received. Subsequently, the user can determine the next step of the therapy treatment based on the response from the patient, whether to continue the treatment or stop the treatment. In some embodiments, the treatment may be altered with different sound frequencies or tones, or used in combination with other stimulus (e.g., combine sound with light, or with tactile).

Disclosed are system and method for delivering therapy treatment to help people release emotions and sensations attached to disturbing memories. The system and method can be used in conjunction with web-based or mobile-based application to provide horizontal eye movement or bilateral auditory or tactile stimulation to process out the emotional disturbances, specifically, using the application together with headphones, light bars or tactile objects. In some instances, sensors and monitors including blood pressure, heart rate, breathing rate and body temperature may be incorporated in conjunction with tactile, visual or auditory devices.

In one aspect, the present invention features a system for providing a mental health therapy treatment. The system can include a display, at least one electronic device that can provide a stimulus to a patient, at least one processor, and at least one memory that includes computer program code. The memory and the computer program code can be configured to interface with the processor to cause the system to perform various operations including: (1) displaying, on the display, a graphical user interface having multiple selectable icons that can be configured to manipulate at least one of the electronic devices, and (2) initiating at least one of the electronic devices to deliver a stimulus associated with the electronic device.

One or more of the following features may also be included. The operations performed can further include monitoring a patient that is receiving the stimulus from at least one of the electronic devices. The operations can include analyzing a response of the patient receiving the stimulus. The operations performed can include adjusting the delivered stimulus based on the response from the patient. The selectable icons can be configured to control sounds, frequency, tones, volume, visual stimulations, auditory stimulations, or any combination thereof. The stimulus can include lights, sounds, volume variations, images, tactile objects, or any combination thereof. The electronic devices can include headphones, light bars, tactile objects, or any combination thereof. The electronic devices can include a blood pressure sensor, heart rate sensor, breathing rate sensor, body temperature sensor, or any combination thereof. The electronic devices can be communicatively coupled to the processor by wired or wireless connection. The system can be configured as a web-based application or software application operating on a computer system or mobile device including desktop computers, laptops, cell phones, and portable tablets. The system can be coupled to at least one external electronic device.

In another aspect, the present invention features a method of providing a mental health therapy treatment. The method can include a system having a display, at least one electronic device that can provide a stimulus to a patient, at least one processor, and at least one memory that includes computer program code. The memory and the computer program code can be configured to interface with the processor to cause the system to perform various operations. The method can include: (1) displaying a graphical user interface having multiple selectable icons that can be configured to manipulate at least one of the electronic devices, and (2) initiating at least one of the electronic devices to deliver a stimulus associated with the electronic device.

One or more of the following features may also be included. Monitoring a patient receiving the stimulus from at least one electronic device. Analyzing a response of a patient receiving the stimulus. Adjusting the delivered stimulus based on the response from the patient. Controlling sounds, frequencies, tones, volume, visual stimulations, auditory stimulations, or any combination thereof delivered to the patient. Delivering a stimulus utilizing light stimuli, sound stimuli, volume variation, image stimuli, tactile objects, or any combination thereof. Communicatively coupling at least one of the electronic devices to the processor via wired or wireless connection. Configuring the system as a web-based application or software application operating on a computer system or mobile device.

These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

Various aspects of at least one example of the present invention are discussed below with reference to the accompanying figures, which are not intended to be drawn to scale. The figures are included as a reference to provide illustration and a further understanding of the various aspects and examples and are incorporated in and constitute a part of this specification but are not intended as a definition of the limits of the disclosure. In the figures, identical or nearly identical components illustrated in various figures may be represented by a like reference character or numeral. For purposes of clarity, not every component may be labeled in every figure.

FIG. 1 is an embodiment of a user interface for a therapy treatment application.

FIG. 2 is a cross-section of a brain showing how differing frequencies of tones used in the therapy treatment can affect different parts of the brain.

FIG. 3 is a flowchart of a method of using the therapy treatment application.

FIG. 4 is an embodiment of a user interface for a therapy treatment application.

FIG. 5 is an embodiment of a user interface for a therapy treatment application.

FIGS. 6A-6B show a user interface for a therapy treatment application with different sides of a headphone being activated.

FIG. 7 is a flowchart of a method of how a user can set up and use a B-LSS application.

FIG. 8 illustrates an embodiment of the therapy treatment application.

FIG. 9 is a flowchart of a method of utilizing the therapy treatment application.

### TERMINOLOGY & NON-LIMITATIONS

Examples of the systems, methods and apparatuses discussed herein are not limited in application to the details of construction and the arrangement of components set forth in the following description or illustrated in the accompanying drawings. The systems, methods and apparatuses are capable of implementation in other examples and of being practiced or of being carried out in various ways. Examples of specific implementations are provided herein for illustrative purposes only and are not intended to be limiting. In particular, functions, components, elements, and features discussed in connection with any one or more examples are not intended to be excluded from a similar role in any other examples.

Examples disclosed herein may be combined with other examples in any manner consistent with at least one of the principles disclosed herein, and references to “an example,” “some examples,” “an alternate example,” “various examples,” “one example” or the like are not necessarily mutually exclusive and are intended to indicate that a particular feature, structure, or characteristic described may be included in at least one example. The appearances of such terms herein are not necessarily all referring to the same example.

Also, the phraseology and terminology used herein is for the purpose of description and should not be regarded as limiting. Any references to examples, components, elements, acts, or functions of the computer program products, systems and methods herein referred to in the singular may also embrace embodiments including a plurality, and any references in plural to any example, component, element, act, or function herein may also embrace examples including only a singularity. Accordingly, references in the singular or plural form are not intended to limit the presently disclosed systems or methods, their components, acts, or elements. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms.

### DETAILED DESCRIPTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form to facilitate describing the present invention.

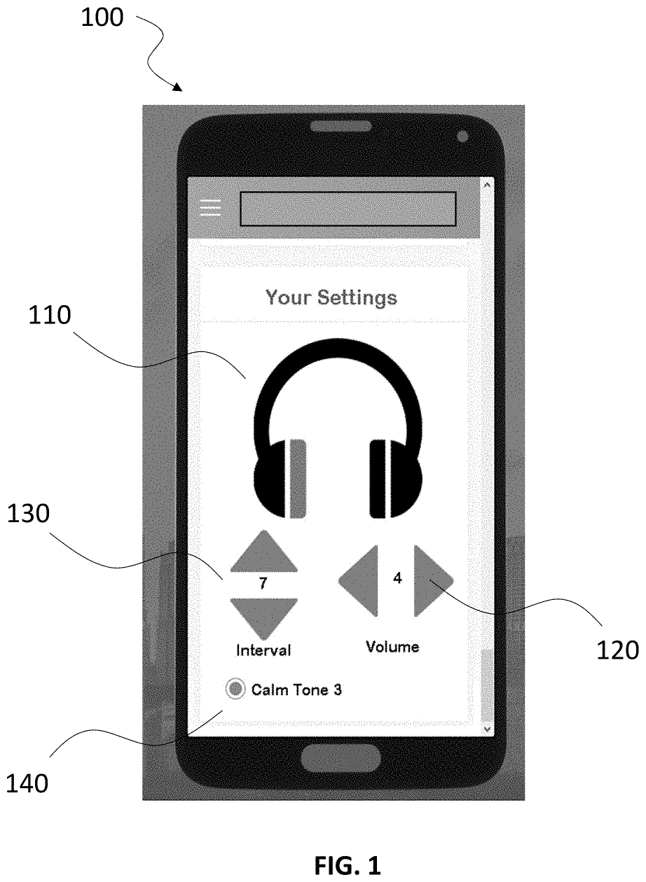

FIG. 1 shows an embodiment of a user interface 100 of a software application operating on a mobile device to allow a user to utilize such application for therapeutic purposes, for example, for treating the user personally, or for treatment of a patient by the user using the application. Although the user interface as shown is for a mobile application, it will be understood that similar user interfaces may be used on various computer platforms and accessed via the Internet and web browsers.

As shown, the user interface 100 may include a headset controller 110 showing which channel (e.g., side) of the headset is active (e.g., graphical indication of which audio channel is currently playing). It is understood that the headset (not shown) may be coupled to the electronic device accessing the application via wired or wireless connection. For example, left channel (e.g., left side) of the headset may be turned off such that the left ear will receive no auditory input. In the alternative, right channel (e.g., right side) of the headset may be turned off such that the right ear will receive no auditory input. In one embodiment, both channels may be active and turned on. In another embodiment, both sides of the headset may be turned off leaving only the other forms of stimulation, e.g., tactile or visual, which will be discussed below. In some embodiments, other types of sensors and monitors (not shown) including blood pressure, heart rate, breathing rate and body temperature may be incorporated in conjunction with tactile, visual or auditory tools, whereby all the devices may be controlled or monitored by the application. The illustrated image shows the left side of the headset being active represented by visual differentiation, but other forms of differentiation may be used.

The user interface 100 also includes a volume controller 120 for controlling sound intensity to the headset playback device. The sound coming out of the headphone can include beeps, tones, metronomes, music or any other types of sounds, whether at regular or random intervals. In some embodiments, the volume and length of intervals between tones can be controlled or adjusted (e.g., longer intervals or pauses, shorter intervals or pauses) to enhance the treatment process with an interval controller 130. In some embodiments, the playing tones may also alternate between left and right channels on the headset playback device, or at varying intervals as determined by the application. The controllers as shown may be operated manually by the user or automatically by the application. If automatically, programs may be designed and saved according to user preference or specific to patient need and recalled via a program controller 140.

As each patient reacts differently to the treatment method, the application can be interactive and adapted for different special needs population. In addition to the physical attributes, other factors and variables to take into consideration during the treatment process include family dynamics and other creative interventions such as dictating or controlling patient behavior (e.g., asking the patient to meditate at a certain hour of the day or sleep at a certain hour of the night), or modifying the way the patient in-takes information (e.g., asking the patient to read the information out loud or write the information on paper together with reading it out loud). Other cognitive processing that can be incorporated in conjunction with the application and the treatment method includes making the patient more aware of his or her behavior and how he/she transforms such information, stores such information, process and recovers such information, and how such information is put to work or can be put to work.

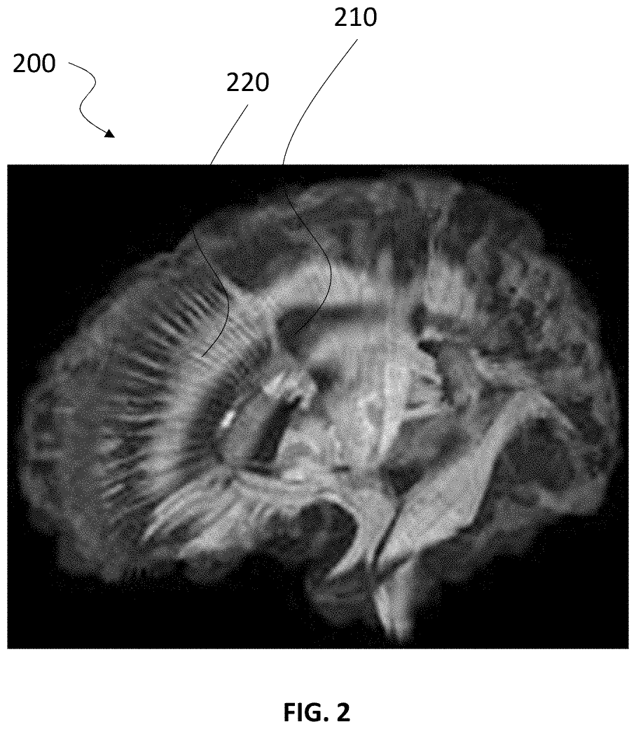

FIG. 2 shows an embodiment of a cross-section of a brain 200, whereby differing frequencies of tones used in the therapy treatment can affect different parts of the brain. For example, interval tones and volume intensities may affect a first portion of the brain 210. In some instances, the frequency of the sound may also be a contributing factor to affecting different parts of the brain. For example, lights and other visual stimulations may affect a second portion of the brain 220. In general, stimulating the neural regulation of the middle ear muscles may be linked to the neural regulation of facial expression, including looking, listening and vocalizing by the individual. In accordance with this application and stimulation of different parts of the brain, neurological stimulation can benefit speech communication, reduce self-regulation, and improve social skills, thereby providing the therapy treatment.

To further support these features, the application may utilize pre-stored soundtracks of tones or frequencies. In some embodiments, the choice of playback tones or frequencies, e.g., pre-stored soundtracks, may include sample MP3's or tones of varying hertz (Hz) in a catalog library. In other embodiments, the choice of playback tones or frequencies may be recorded by the user or a third-party and imported into the application. In yet another embodiment, the soundtracks may include video tracks in movie or short clip form. There may also be audio or visual feedbacks in view of the playback tones or frequencies.

In one embodiment, the application may be able to receive feedback from the target patient in a controlled fashion. For example, a user may be able to provide feedback of the playback tones or frequencies in an anonymous fashion by providing comments via a secure web link. This can be done on the Internet or via a mobile device. In the alternative, the application may be able to collect user/patient information, with user's/patient's consent, in order to gather more data and/or to produce additional playback tones or frequencies for the treatment of others.

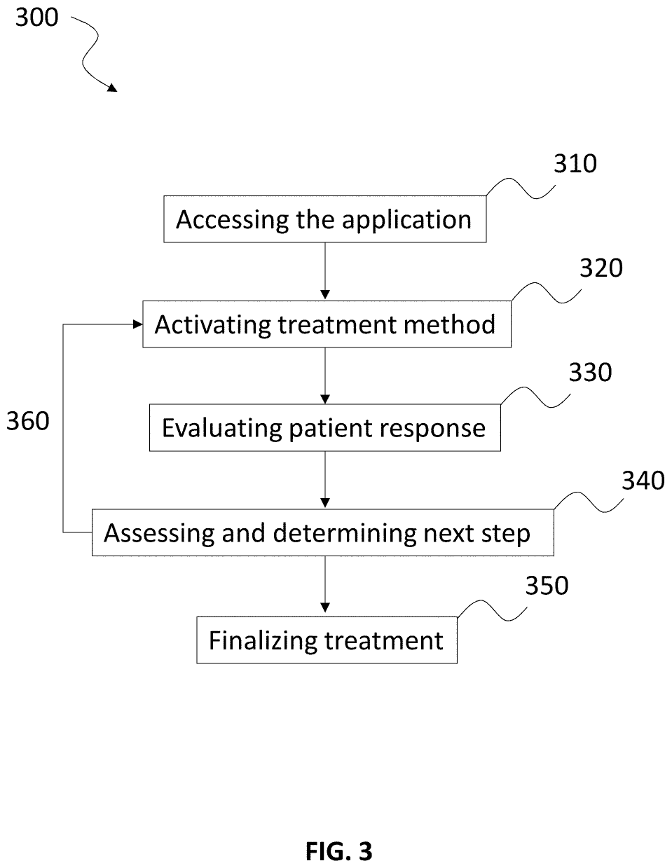

FIG. 3 is a flowchart 300 of a method of using the therapy treatment application as described above. In one embodiment, the method starts with a user accessing the application 310 similar to those described above, whether via a laptop, mobile device, or other suitable electronic devices. The user may be accessing the application for the purpose of treating him/herself, or for the treatment of others. In general, the treatment application is intended to self-sooth trauma patients by mimicking rapid eye movement with sounds or other external visual or auditory stimulations.

Next, once accessed, the user can activate the treatment method 320. The treatment method may include stimulating the patient with sound or frequency, visual or tactile techniques. For example, rapid eye movements can be mimicked with interactive sounds (e.g., beeps) and volume. In other words, using sounds of differing frequency or volume can help to desensitize and reprocess a patient's eye movement such that he/she feels relaxed and soothed as if entering a light stage of sleep. In some instances, the activation step 320 may include visual interactions such as lights on display using an external light source like a light bar (not shown). In other instances, the activation step 320 may include tactile stimulations via interactive toys, gloves and other suitable objects capable of tactile controls and adjustments (not shown). As above, the external devices such as the light source and the tactile stimulation objects may be coupled to the application via known wired or wireless techniques. In some embodiments, the activation step 320 may include other sensors such as blood pressure, heart rate, breathing rate and body temperature monitors (not shown), which can also be coupled to the application via known connection techniques.

In one embodiment, the activation step 320 may include using a set of headphones and controlling such headphones using the steps described above so as to block out any and all ambient noise. And like that discussed above, the activation step 320 can be manually adjusted via the interactive application as described above, or may be pre-loaded with a saved program setting. The adjustments can include adjusting the timing of the sound on the headphone, the frequency of the sound, as well as the lights on display, among other variables.

After the stimulus has been activated, the next step is evaluating the patient's response 330. This response will be based on the area of the brain that is activated or stimulated in accordance with the applied stimulus, e.g., frequency, sound, light or tactile. The evaluation step may be done with known scanning techniques on the patient's brain, or by examining the patient's mental and physical responses to the earlier activated stimulus. In some embodiments, some stimulus may be more effective than others. For example, sound at differing frequencies may be more effective than light or tactile stimulation. In other embodiments, light or tactile stimulation may further enhance the sound treatment. The patient's response may be evaluated manually by a medical professional or may be monitored by the patient him/herself.

Next, assessing and determining the next step 340 includes whether to add or change the stimulus. For example, going to a sound level with higher frequency to see if that helps to stimulate a different part of the patient brain so the patient is more relaxed or soothed. In the alternative, lower the frequency to see stimulate a different part of the patient brain to facilitate relaxation. In some instances, if the patient is feeling better, then the treatment process may end thus leading to finalizing the treatment step 350.

If, for example, there is a need to subject the patient to a sound level with higher or lower frequencies, or different intervals (e.g., beeps or sounds), then the user may revisit and repeat the activating step by going back to the activating treatment method 320 and reapplying the stimulus, whether the same stimulus or a different stimulus, or a combination of stimuli.

When processes are represented or implied in the block diagram, the steps may be performed by one element or a plurality of elements. The steps may be performed together or at different times. The elements that perform the activities may be physically the same or proximate one another or may be physically separate. One element may perform the actions of more than one block.

In one embodiment, disclosed is a system for providing a therapeutic benefit to calm and regulate the nervous system in an accessible form of an electronic device one can use at home. This allows convenience for therapist to provide online therapy and for client access to therapy and use for self. In one instance, the provider may guide the client use on a mobile or electronic device with access to Wi-Fi when downloaded. In another instance, the provider may direct client about speed and length of time with processing cognitive information related to emotional processing associated with targeted issue. In operation, the client will receive auditory stimulation for a duration of a period and re-evaluating increase in speed to activate neural activity or reduce speed to calm and integrate process. In some embodiments, the client may be the actual user or recipient of the treatment. In other embodiments, the client may be the guardian of the actual user or recipient of the treatment. The neural activity may be stimulated by auditory sounds and will continue after the session and neural processing will continue based on the physiological response. When used as a therapeutic intervention there may be other techniques and protocols one can use for cognitive processing.

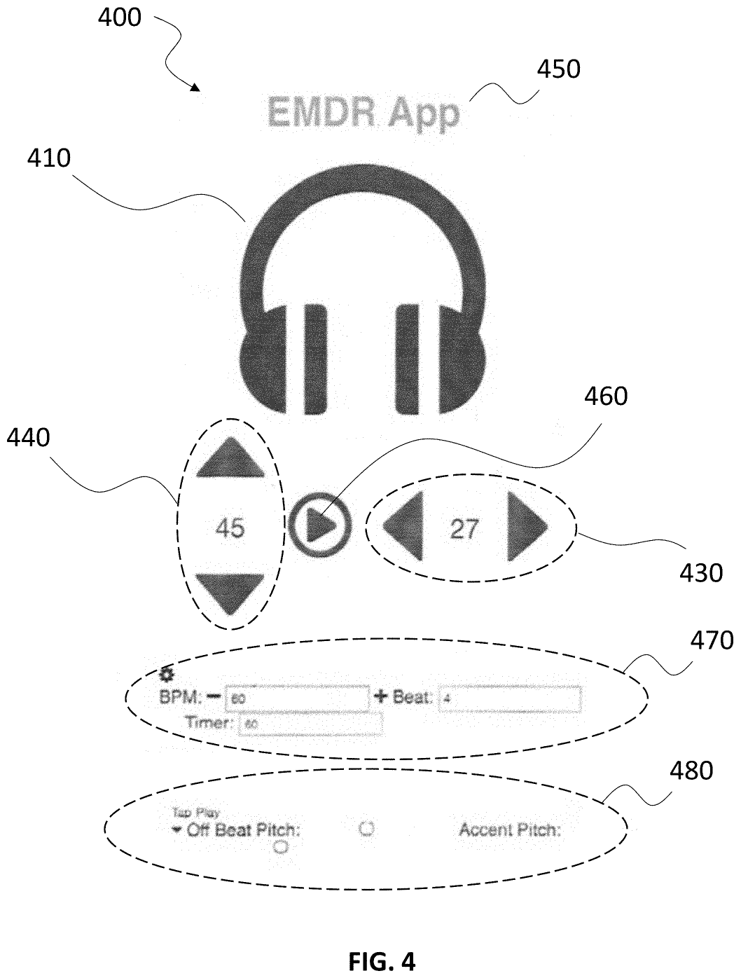

FIG. 4 discloses a GUI for a Bi-Lateral Sound Stimulation (B-LSS) application 450 for a treatment system 400. The B-LSS application 450 can be used to activate and integrate information from the two hemispheres of a brain and used as a tool to process cognitive information for EMDR as a psychotherapeutic intervention. In operation, Bi-Lateral Stimulation (BLS) mimics Rapid Eye Movements (REM) during dream stage in sleep in an auditory rhythmic alternating pattern and provides a therapeutic benefit to recipient. BLS is a component used in EMDR and brain spotting for therapy intervention to reduce distressing memories.

The benefits of the disclosed system 400 include stimulating neural activity via alternating bi-lateral sounds in left and right sides of the head, regulating the central nervous system by relaxing heart rate and blood pressure, and release both physiological and emotional sensations with cognitive processing, to name a few. Additionally, by using the disclosed system 400 a user will be able to calm down and regulate his or her nervous system, reduce anxiety, improve focus and attention, improve speech communication, improve sleep by calming the nervous system and assist in falling and staying asleep, and activate and synch the brain waves of overconnectivity and underconnectivity of the sensory system.

In operation, the B-LSS application 450 can function with at least the following functions and controls: sound, speed, volume, on/off/pause, and headphones to provide visual stimulation, among others.

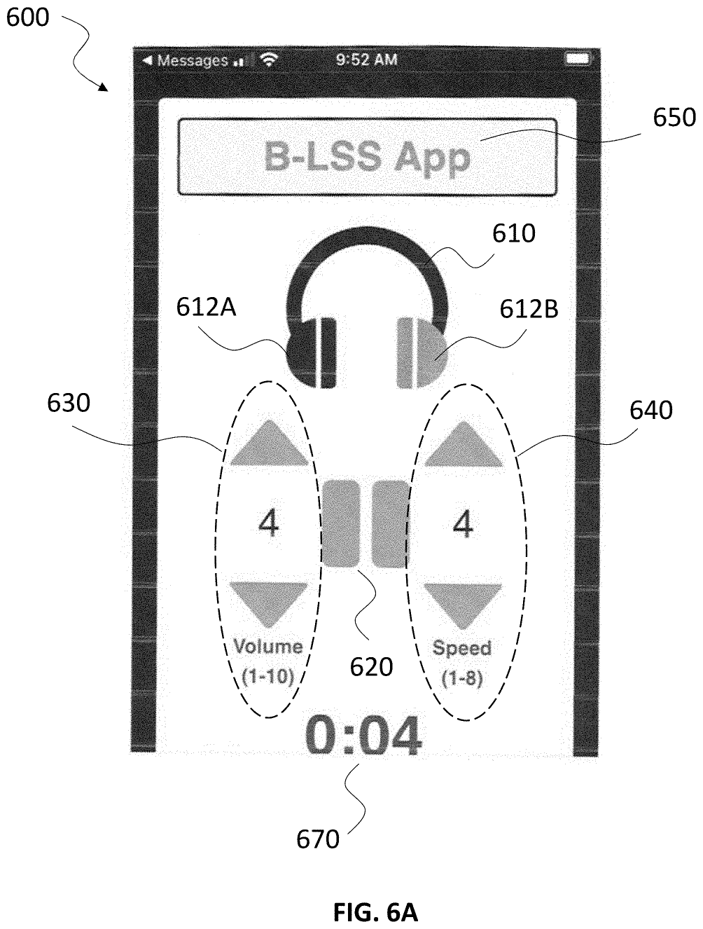

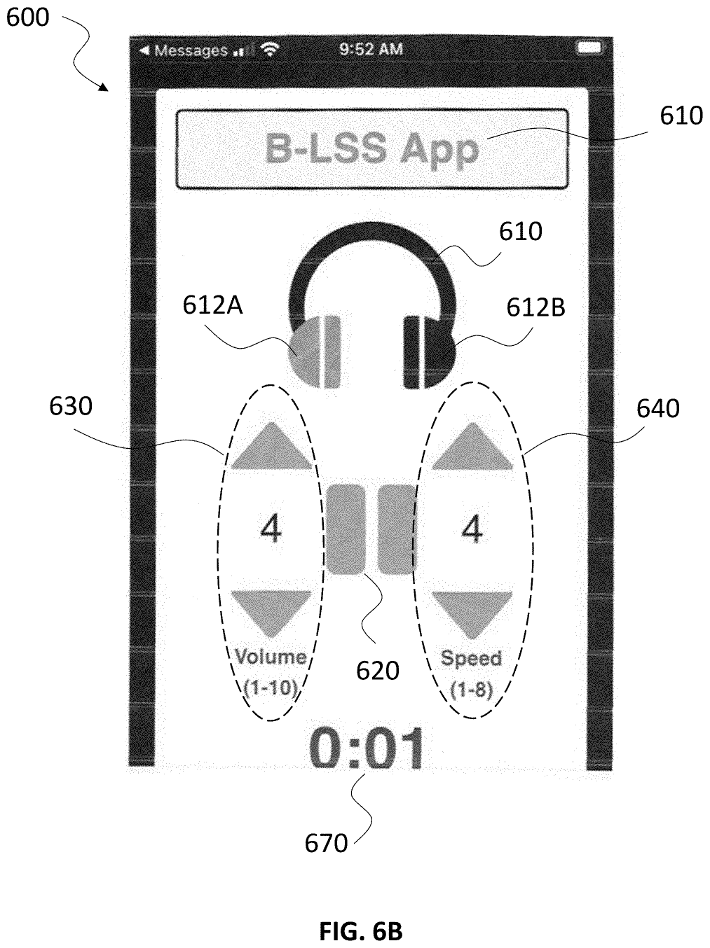

As shown in FIG. 4, the graphical user interface (GUI) of the B-LSS application 450 may include a headset controller 410 similar to that described above. The headset controller 410 may provide a graphical indication of which audio channel is currently active as best illustrated in FIGS. 6A-6B and will be described and discussed in more detail below.

In one embodiment, the GUI may also include a volume controller 430 similar to that discussed above for controlling volume intensity to a headset playback device. In operation, the volume intensity may be increased by actuating the right arrow and decreased by actuating the left arrow. In some embodiments, a value indicator (e.g., 27) may be displayed between the two arrows to provide a user with an indication of the strength of the sound being delivered to the headset playback device.

In one embodiment, the GUI may also include a speed controller 440 for controlling the speed of the sound delivered to a headset playback device. In operation, the speed can be increased by actuating the up arrow and decreased by actuating the down arrow. In some instances, a value indicator (e.g., 45) may be displayed between the two arrows to provide a user with an indication of the speed of the sound being delivered to the headset playback device.

In one embodiment, the GUI may also include a start/stop/pause button 460 located between the volume controller 430 and the speed controller 440. This central button 460 may be actuated to alternate among starting the B-LSS application 450, stopping the B-LSS application 450, or pausing the B-LSS application 450.

In one embodiment, the GUI may further include a physical parameters panel 470 displaying, for example, a user's blood pressure per minute (BPM), heartbeat, and a timer for measuring or counting purposes. In some embodiments, the physical parameters panel 470 may include other types of user's physical parameters as described above. In these instances, the B-LSS application 450 need to be in wireless communication with external electronic devices for receiving the physical parameter measurements from such external electronic devices, so as to be able to display the physical parameters in real-time. This will be discussed in more detail below.

In some embodiments, the B-LSS application 450 will be in communication with external sensors and monitors including, for example, an electronic watch that is in wireless communication with the user's mobile device or laptop computer running the B-LSS application 450. The methods of wirelessly coupling these electronic devices are known by one skilled in the art and will not be elaborated further herein. Once in wireless communication, the B-LSS application 450 may be in integral communication with the external device (e.g., wireless watch, heart monitor) which may be attached to parts of the user's body (e.g., arm, chest). As these external devices are attached to the user's body and are carrying out the physical measurements (e.g., heartbeat, heart rate, pulses), these measurements and the associated data may then be wirelessly communicated from the external devices to the B-LSS application 450 and displayed accordingly on the GUI. In the alternative, the data may be stored on a database or server hosting the B-LSS application 450.

In other embodiments, the GUI may include a sound pitch controller 480. In this example, the sound pitch controller 480 allows a user to adjust the type of pitches being played on a headset playback device. For instance, a user may choose to play a pitch that is offbeat or having an accent. The user may also choose other types of pitches that may help toward providing the therapeutic effects. The pitches may be pre-stored on a database or subsequently uploaded to the B-LSS application 450.

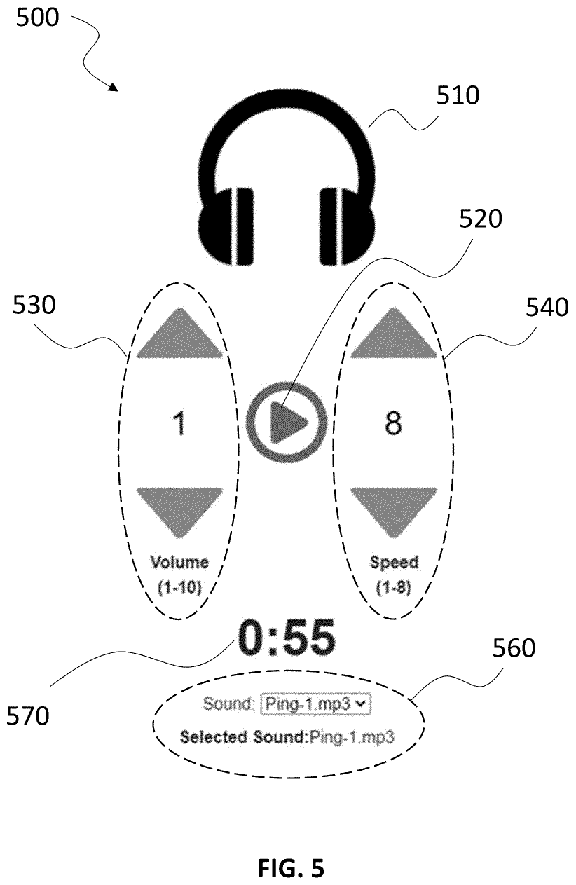

FIG. 5 discloses another GUI associated with a B-LSS software application 500 according to another embodiment of the present invention. In this embodiment, the GUI includes a headset controller 510 similar to those described above and will not be elaborated further herein. Similarly, the GUI may also include a start/stop/pause button 520, a volume controller 530 and a speed controller 540 similar to those described above and will not be elaborated further herein.

In one embodiment, the B-LSS application 500 may include a pre-determined or pre-selected sound controller 560. As shown in the figure, the user is playing the Ping-1.mp3 soundtrack. This soundtrack may provide particularly effective therapeutic effects to a user. In other embodiments, the user may play other types of soundtracks. These soundtracks may be uploaded to the B-LSS application 500 by the user or by the therapist. In the alternative, there may be a plurality of therapeutic soundtracks pre-stored on the server hosting the B-LSS application 500, which may subsequently be provided or offered to a user via the B-LSS application 500.

In another embodiment, the B-LSS application 500 may also include a timer 570. The timer 570 may display the amount of time that has progressed in the current treatment session. The timer 570 may serve to help a user keep track of how long he or she has been using the B-LSS application 500 and allow a user to know when to stop and limit the duration of treatment (as necessary). In the alternative, the timer 570 may help a user determine that an insufficient amount of time has lapsed, and that the user should continue with the treatment process or give the treatment process sufficient amount of time in order for the treatment to be successful. Upon actuation of the start/stop/pause 520 button the timer 570 will react accordingly. In other words, the timer 570 will start counting once the start/stop/pause button 520 has been actuated, and pause or stop when the start/stop/pause button 520 is actuated again.

FIGS. 6A and 6B show a user interface 600 for a therapy treatment application 650 with different sides of a headphone 612A, 612B being activated. Similar to above, the B-LSS application 600 includes a headset controller 610, a start/stop/pause button 620, a volume controller 630, a speed controller 640, and a timer 670 similar to those described above and will not be elaborated further herein.

As shown, the headset controller 610 includes left headphone 612A and right headphone 612B. FIG. 6A shows that the right headphone 612B (gray in color) has a different color indicator than the left headphone 612A (black in color). Likewise, FIG. 6B shows that the left headphone 612A (gray in color) has a different color indicator than the right headphone 612B (black in color). The different color indicator may be used as a graphical indication of which audio channel is currently playing. For example, in FIG. 6A the right headphone 612B is active (gray) and sound is coming out of this side of the headset while the left headphone 612A is inactive (black) and no sound is coming out of this side of the headset. Conversely, the opposite is true in FIG. 6B. The ability to turn on/off different sides of the headset will further help to provide different forms of auditory stimulation to different parts of the brain for therapeutic treatment purposes. In one embodiment, both left and right channels (gray) of the headset may be turned on and active in delivering sound to both ears.

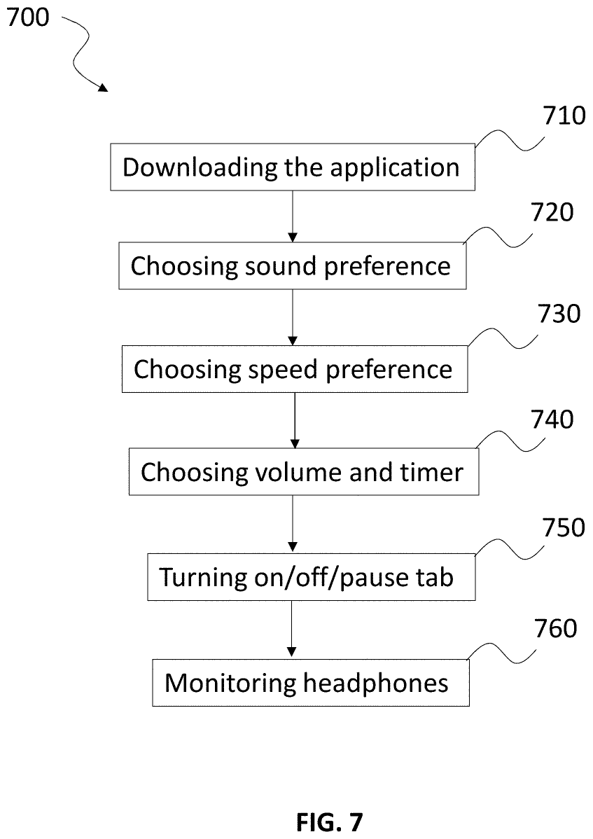

FIG. 7 is a flowchart 700 of a method of how a user can set up and use the B-LSS application 450 as described above. In one embodiment, the method starts with a downloading step 710 where the user downloads the application 450. The B-LSS application 450 can be downloaded from an accessible website/webpage. In the alternative, a download link may be provided to the B-LSS application 450 via an email or provided on the website/webpage. In yet another alternative, the B-LSS application 450 can be downloaded via an “app store” from a user's mobile device. It will be understood and appreciated by one skilled in the art that there are many ways to download or access the B-LSS application 450 either via a laptop or a mobile, electronic device.

Next, once the B-LSS application 450 has been installed on the electronic device, the user can choose the sound preference in step 720. In choosing the sound preference step 720, the user can tap a start/stop or on/off button similar to those described above (e.g., 460, 520, 620). In one embodiment, the on/off button 460 can be in the center adjacent to volume controls 420 and speed controls 430. The choice of sound can be selected by tapping on the sound choices below a timer similar to those describe above (e.g., 480, 560). For example, there may be the option to choose sound such as Ping 1, Ping 3 or Ping 5 similar to those discussed above (e.g., Ping 1 in FIG. 5). Tapping the desired sound choice button once will start playing the sound of choice and tapping the button again will cause the desired sound choice to stop.

Next, the user can choose the speed preference in step 730. In choosing the speed, the user can tap an “up arrow” to increase the speed or a “down arrow” to decrease the speed similar to those discussed above (e.g., 440, 540, 640). In one embodiment, the speed can range from a scale of 0 to 8, with 0 being the slowest speed and 8 being the fastest speed. While only values ranging from 0 to 8 are discussed herein, it will be appreciated by one skilled in the art that that there can be different ranges of speeds with different values or abbreviations for describing the variations in speeds.

Next, the user can choose the volume and timer in step 740. In choosing the volume, the user can tap buttons similar to those for the speed (e.g., 430, 530, 630), where one button increases the volume, and another button decreases the volume. In one embodiment, the volume can range from a scale of 0 to 10, with 0 being silent and 10 being the loudest. For proper treatment a user should select the volume choice that is most appropriate for the user. Additionally, the user can pause and continue the application by actuating the start/stop or on/off button as described before. In this step, a timer may be displayed below the above controls (e.g., 570, 670). The timer helps to act as a guide for the amount of time the program has been activated and to track the duration of use. Like with the speeds, while only values ranging from 0 to 10 are discussed herein, it will be appreciated by one skilled in the art that that there can be different ranges of volume intensity with different values or abbreviations for describing the variations in volume intensity.

While the options described in steps 720, 730, 740 have been described in the order discussed above and shown in FIG. 7, it will be appreciated and understood by one skilled in the art that a user need not follow these steps in the exact order and that the user can follow different orders of these steps. For example, steps 730 and/or 740 can take place before step 720, while step 720 can take place between steps 730 and 740, among others.

Next, once all the options described in steps 720, 730, 740 have been selected and determined, a user can, in step 750, turn on the application, turn off the application, or stop the application by actuating the on/off or start/stop button as discussed above. In other words, the user can activate the application by tapping the start button to turn on, off or pause for the duration needed.

Next, as the application is running, paused or turned off, the user can check on the headphones in step 760. In one embodiment, the headphones can provide visual stimulation in the application by changing the color of the headphones similar to those described above (e.g., 612A, 612B). By lighting up a different color (e.g., blue v. black) in alternating left and right, the visual stimulation provides a way to indicate synching of the sounds corresponding with either being auditory on the left or right side of the ear.

The disclosed embodiments of the B-LSS application as disclosed herein can be set up along with the following recommendations and benefits. Begin by turning on and listening to the bi-lateral sounds so the brain will adjust to the synch of left side and right side of the auditory stimulation. It is more effective if using with headphones due to location closer to ears activating the auditory sensory system. If a user is unable to tolerate the headphones, start with BLS behind the person's head and centered. After first use, the brain will begin to sync with REM especially when eyes are closed. A user may notice eyeballs twitching left and right to the sounds. Approximately 3-5 minutes a user may notice their body and breathing relaxing. In some instances, some may feel a “drop” in the body which tends to indicate relaxing. If a user has control issues, he or she may not relax as quickly and redirect with an imagery of an activity they enjoy. A user should check their breathing rate by noticing their chest rising and dropping this tends to indicate it is calming them. In some instances, reports of people saying they feel relaxed and can feel like they want to fall asleep, further supports activation of REM sleep.

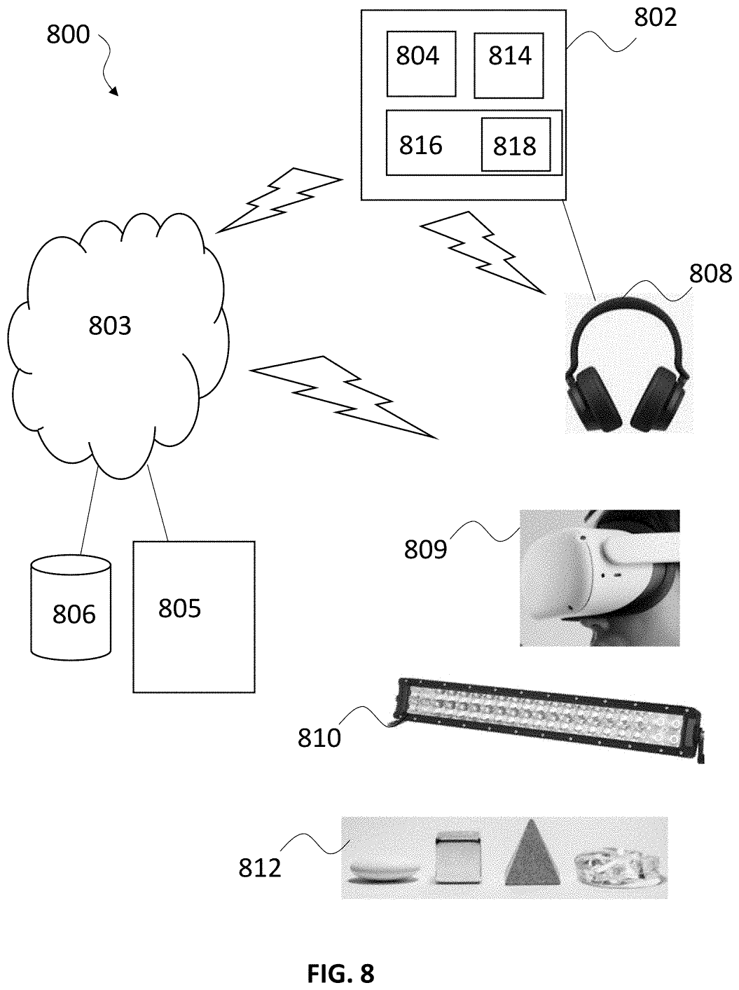

Referring to FIG. 8, in one aspect, the present invention features a system 800 for providing a mental health therapy treatment. The system 800 can be configured as a web-based software application or other software application operating on a computer platform (e.g., desktop, laptop, tablet) or mobile device (e.g., cellphone) 802. The system 800 can be configured to operate locally as a standalone system, or as a distributed system that can operate remotely via the internet or other networked environment 803.

The system 800 can include a display 804, a server 805, a data store 806 (i.e., local or remote), and at least one electronic device such as headphones 808, visual headgear 809, and light bars 810 that can provide a stimulus to a patient. The headphones 808 can provide auditory stimulation having various tones, frequencies, and volume. The visual headgear 809 can provide light and image stimulation of varying intensity. The light bar 810 can provide a patient with varying light stimulation (e.g., strobe). In some embodiments, tactile objects 812 can be included to provide a patient with tactile stimulation. In some embodiments, the system 800 can include sensors and monitoring devices for monitoring a patient's blood pressure, heart rate, breathing rate, and body temperature, while the patient is receiving treatment.

The electronic devices 808, 809, 810 can be utilized in combination when delivering a therapy treatment or they can be utilized individually. As shown in FIG. 8, the electronic devices 808, 809, 810 are configured as external devices in relation to the user's computer platform or mobile device 802, however, in other embodiments visual and auditory stimulation can be delivered directly on the display 804 of the user's platform 802.

The system 800 can further include at least one processor 814, and at least one memory 816 that includes computer program code 818 for the software application. The processor 814 can be implemented in software, firmware, or a combination of hardware and software. The memory 816 and the computer program code 818 can be configured to interface with the processor 814 to cause the system 800 to perform various operations including: (1) displaying, on the display 804, a graphical user interface having multiple selectable icons (as shown in FIGS. 1, 4, 5, 6A, 6B) that can be utilized to manipulate at least one of the electronic devices 808, 809, 810; (2) initiating at least one of the electronic devices 808, 809, 810 to deliver a stimulus associated with the electronic device; (3) monitoring a patient that is receiving the stimulus from at least one of the electronic devices 808, 809, 810; (4) analyzing a response of the patient receiving the stimulus; and (5) adjusting the delivered stimulus based on the response from the patient.

The selectable icons on the GUI presented on the display 804 can be configured and used to control sounds, frequency, tones, volume, visual stimulations, auditory stimulations, or any combination thereof. The stimulus that is applied to a patient can include lights, sounds, volume variations, images, tactile objects, or any combination thereof. In various embodiments, the electronic devices 808, 809, 810 can be communicatively coupled to the system 800 by wired or wireless connection.

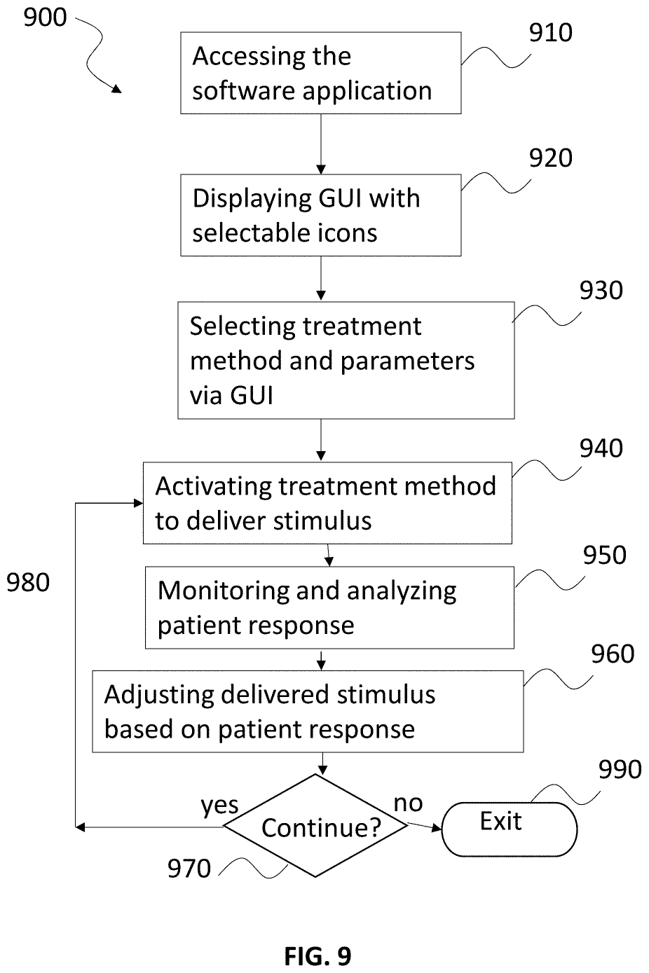

Referring to FIG. 9, in another aspect, the present invention features a method of providing a mental health therapy treatment. The method (900) can be implemented on and utilized with the system illustrated in FIG. 8 and discussed above. The method (900) starts (910) by launching and accessing a software application. Once launched, the application displays (920) a GUI with selectable icons that can be utilized for selecting (930) a particular treatment option. The method (900) proceeds by activating (940) a selected treatment option to deliver a stimulus to a patient via one or more electronic devices. The patient response can be monitored and analyzed (950) while the stimulus is applied to the patient. Based on the patient response, the stimulus can be adjusted (960) accordingly. The method (900) can then decide (970) whether to continue applying the treatment, by looping (980) through steps (940), 950), (960), or, in the alternative, to terminate the treatment and exit (990) the software application.

### Alternative Configurations and Implementations

In this description, various functions and operations may be described as being performed by or caused by software code to simplify description. However, those skilled in the art will recognize what is meant by such expressions is that the functions result from execution of the code by a processor, such as a microprocessor. Alternatively, or in combination, the functions and operations can be implemented using special purpose circuitry, with or without software instructions, such as using Application-Specific Integrated Circuit (ASIC) or Field-Programmable Gate Array (FPGA). Embodiments can be implemented using hardwired circuitry without software instructions, or in combination with software instructions. Thus, the techniques are limited neither to any specific combination of hardware circuitry and software, nor to any particular source for the instructions executed by the data processing system.

While some embodiments can be implemented in fully functioning computers and computer systems, various embodiments are capable of being distributed as a computing product in a variety of forms and are capable of being applied regardless of the particular type of machine or computer-readable media used to actually effect the distribution.

At least some aspects disclosed can be embodied, at least in part, in software. That is, the techniques may be carried out in a computer system or other data processing system in response to its processor, such as a microprocessor, executing sequences of instructions contained in a memory, such as ROM, volatile RAM, non-volatile memory, cache or a remote storage device.

Routines executed to implement the embodiments may be implemented as part of an operating system or a specific application, component, program, object, module or sequence of instructions referred to as “computer programs.” The computer programs typically comprise one or more instructions set at various times in various memory and storage devices in a computer, and that, when read and executed by one or more processors in a computer, cause the computer to perform operations necessary to execute elements involving the various aspects.

A tangible, non-transitory computer storage medium can be used to store software and data which when executed by a data processing system causes the system to perform various methods. The executable software and data may be stored in various places including for example ROM, volatile RAM, non-volatile memory and/or cache. Portions of this software and/or data may be stored in any one of these storage devices. Further, the data and instructions can be obtained from centralized servers or peer to peer networks. Different portions of the data and instructions can be obtained from different centralized servers and/or peer to peer networks at different times and in different communication sessions or in a same communication session. The data and instructions can be obtained in entirety prior to the execution of the applications. Alternatively, portions of the data and instructions can be obtained dynamically, just in time, when needed for execution. Thus, it is not required that the data and instructions be on a machine-readable medium in entirety at a particular instance of time.

Examples of computer-readable media include but are not limited to recordable and non-recordable type media such as volatile and non-volatile memory devices, read only memory (ROM), random access memory (RAM), flash memory devices, floppy and other removable disks, magnetic disk storage media, optical storage media (e.g., Compact Disk Read-Only Memory (CD ROMS), Digital Versatile Disks (DVDs), etc.), among others. The instructions may be embodied in digital and analog communication links for electrical, optical, acoustical or other forms of propagated signals, such as carrier waves, infrared signals, digital signals, etc.

In general, a machine readable medium includes any mechanism that provides (i.e., stores and/or transmits) information in a form accessible by a machine (e.g., a computer, network device, personal digital assistant, manufacturing tool, any device with a set of one or more processors).

In various embodiments, hardwired circuitry may be used in combination with software instructions to implement the techniques. Thus, the techniques are neither limited to any specific combination of hardware circuitry and software nor to any particular source for the instructions executed by the data processing system.

Although some of the embodiments provide a number of operations in a particular order, operations which are not order dependent may be reordered and other operations may be combined or broken out. While some reordering or other groupings are specifically mentioned, others will be apparent to those of ordinary skill in the art and so do not present an exhaustive list of alternatives. Moreover, it should be recognized that various aspects could be implemented in hardware, firmware, software, or any combination thereof.

For ease of exposition, not every step or element of the systems and methods described above is described herein as part of a computer system, but those skilled in the art will recognize that each step or element may have a corresponding computer system or software component. Such computer system and/or software components are therefore enabled by describing their corresponding steps or elements (that is, their functionality), and are within the scope of the disclosure.

Having described above several aspects of at least one example, it is to be appreciated various alterations, modifications, and improvements will readily occur to those skilled in the art. Such alterations, modifications, and improvements are intended to be part of this disclosure and are intended to be within the scope of the invention. Accordingly, the foregoing description and drawings are by way of non-limiting example only, and the scope of the invention should be determined from proper construction of the appended claims, and their equivalents.

What is claimed is:

1. A system for providing a mental health therapy treatment, the system comprising:
a display;
at least one electronic device configured to provide a stimulus;
at least one processor; and
at least one memory including computer program code;
the at least one memory and the computer program code configured to, with the at least one processor, cause the system to perform operations comprising:
displaying, on the display, a graphical user interface (GUI) having a plurality of selectable icons configured to manipulate the at least one electronic device; and
initiating the at least one electronic device to deliver a stimulus associated with the electronic device.

2. The system of claim 1, wherein the performed operations further comprise monitoring a patient receiving the stimulus from the at least one electronic device.

3. The system of claim 1, wherein the performed operations further comprise analyzing a response of a patient that received the stimulus from the at least one electronic device.

4. The system of claim 3, wherein the performed operations further comprise adjusting the delivered stimulus based on the response from the patient.

5. The system of claim 1, wherein the selectable icons are configured to control at least one of sound, frequency, tones, volume, visual stimulations, auditory stimulations, or any combination thereof.

6. The system of claim 1, wherein the stimulus comprises at least one of light, sound, volume variation, images, tactile objects, or any combination thereof.

7. The system of claim 1, wherein the at least one electronic device includes at least one of headphones, light bar, tactile objects, or any combination thereof.

8. The system of claim 1, wherein the at least one electronic device includes at least one of a blood pressure sensor, heart rate sensor, breathing rate sensor, body temperature sensor, or any combination thereof.

9. The system of claim 1, wherein the at least one electronic device is communicatively coupled to the processor by wired or wireless connection.

10. The system of claim 1, wherein the system is configured as a web-based application or software application operating on a computer system or mobile device including desktops, laptops, cell phones, portable tablets.

11. The system of claim 10, wherein the system is coupled to at least one external electronic device.

12. A method for providing a mental health therapy treatment, the method comprising:
in a system having a display, at least one electronic device configured to provide a stimulus, at least one processor, and at least one memory including computer program code, wherein the at least one memory and the computer program code configured to, with the at least one processor, cause the system to perform operations including:
displaying, on the display, a graphical user interface (GUI) having a plurality of selectable icons configured to manipulate the at least one electronic device; and
initiating the at least one electronic device to deliver a stimulus associated with the electronic device.

13. The method of claim 12, further comprising monitoring a patient receiving the stimulus from the at least one electronic device.

14. The method of claim 13, wherein monitoring a patient includes utilizing at least one of a blood pressure sensor, heart rate sensor, breathing rate sensor, body temperature sensor, or any combination thereof.

15. The method of claim 12, further comprising analyzing a response of a patient that received the stimulus from the at least one electronic device.

16. The method of claim 15, further comprising adjusting the delivered stimulus based on the response from the patient.

17. The method of claim 16, wherein adjusting the delivered stimulus includes controlling sound, frequency, tones, volume, visual stimulations, auditory stimulations, or any combination thereof delivered to the patient.

18. The method of claim 12, wherein the delivering a stimulus includes utilizing at least one of light stimuli, sound stimuli, volume variation, images stimuli, tactile objects, or any combination thereof.

19. The method of claim 12, wherein delivering a stimulus includes communicatively coupling the at least one electronic device to the processor via wired or wireless connection.

20. The method of claim 12, wherein displaying the GUI includes configuring the system as a web-based application or software application operating on a computer system or mobile device.
